(19) United States
(12) Patent Application Publication
(10) Pub. No.: US 2020/0386767 A1
(43) Pub. Date: Dec. 10, 2020

(54) Analytical method and apparatus using fingerprints on the basis of types in expression levels of express trace proteins and/or peptides contained in living tissue and/or biological fluid

(71) Applicant: Shinwa Chemical Industries Ltd., Kyoto (JP)

(72) Inventors: Hiroshi Kobayashi, Kyoto (JP); Hiroo Wada, Kyoto (JP); Kazuhiro Imai, Kyoto (JP)

(21) Appl. No.: 16/438,212

(22) Filed: Jun. 11, 2019

(30) Foreign Application Priority Data
Jun. 7, 2019 (JP) 2019-106985

(51) Int. Cl. G01N 33/68; G01N 21/64; A61K 49/00; C07K 1/13; G01N 33/58

(52) U.S. Cl. CPC G01N 33/6848; G01N 21/6428; G01N 33/582; C07K 1/13; A61K 49/0056

(57) ABSTRACT

Provided are an analytical method and apparatus using fingerprints on the basis of the types and expression levels of expressed trace proteins and/or peptides contained in living tissue and/or biological fluid. The present invention relates to a method and apparatus for analyzing the status of living tissue and/or biological fluid by binding a specific fluorogenic reagent (such as DAABD-Cl) to expressed trace proteins and/or peptides contained in living tissue and/or biological fluid without degradation treatment followed by precisely detecting and separating by nano-liquid chromatography, and subsequently continuously and quantitatively measuring the separated and purified fluorescent labelled proteins and/or peptides using a fluorescence detector. The use of a nano-liquid chromatograph having a fluorescence detector makes it possible to obtain profiles of expressed trace proteins and/or peptides contained in living tissue and/or biological fluid.

### BACKGROUND OF THE INVENTION

### Field of the Invention

The present invention relates to an analytical method and apparatus using fingerprints on the basis of the types and expression levels of expressed trace proteins and/or peptides contained in living tissue and/or biological fluid, and more particularly, the present invention relates to a method and apparatus for analyzing the state of living tissue and/or biological fluid by obtaining a so-called fingerprint profile (fingerprint) on the basis of the types and expression levels of proteins and/or peptides expressed in the living body by labelling several thousands to several ten thousands of proteins and/or peptides contained in living tissue and/or biological fluid with a fluorogenic reagent without subjecting to degradation treatment followed by precisely detecting and separating the proteins and/or peptides by nano-liquid chromatography and subsequently continuously and comprehensively quantitatively measuring the separated and purified fluorescent labelled proteins and/or peptides using a fluorescence detector.

The present invention is useful in providing a novel technology and novel apparatus relating to a novel analytical method and apparatus using a fingerprint profile for understanding or controlling the state of living tissue and/or biological fluid or biological activity in the field of proteomics or proteome analysis.

### Background of the Related Art

Proteins contained in living tissue or biological fluid are expressed by the activity of DNA or RNA present in the living body and act as the central component of biological functions or biological activities.

Expression of protein in living tissue is known to be affected by such factors as the environment in which the living tissue is placed even in the case of a single individual having the same DNA.

If it were possible to quantitatively or qualitatively analyze the expression of these proteins contained in the living body, it would be possible to understand the functions of DNA and RNA individually utilized in the living body as well as analyze protein activity that changes according to internal and external factors.

For example, a protein characterized by being the cause of a disease can be discovered by comparing protein expression in living tissue that has become abnormal as a result of that disease with protein expression in living tissue in a so-called normal state followed by analyzing the differences there between.

In addition, by quantitatively or qualitatively analyzing expression of proteins contained in the living body, uses such as control of cell differentiation as intended can be considered by, for example, identifying those characteristic proteins that undergo change before and after cells in a so-called initial state, such as iPS cells, differentiate into various tissues.

In this manner, research involving analysis of proteins expressed in living tissue in order to understand or control the status of the living body or biological activity according to the expression thereof is referred to as proteomics or proteome analysis, and in the field of biomedical or medicinal sciences, for example, research is proceeding on proteins and peptides present in living tissue and/or biological fluid in order to utilize as disease markers relating to diagnosis and treatment by discovering proteins that are related to cancer and other diseases (Non-patent reference 1).

Two techniques consisting of the top-down method and bottom-up method (also referred to as the shotgun method) are widely known in research relating to the above-mentioned proteomics or proteome analysis (Non-patent reference 2).

In the top-down method, all proteins extracted from a cell are injected into the end face of an inert gel plate such as a flat agarose gel plate, and after having fractionating according to molecular size by electrophoresis by applying electricity to the plate, the surface that has been stained with a fluorescent marker is photographed followed by determining expressed proteins on the basis of the luminescence of protein spots, thereby making it possible to separate and measure the amounts of the proteins without having to subject to protein degradation. In the case of proteins undergoing qualitative analysis, the protein spots are analyzed by cutting out the spots while still contained in the gel and analyzing with a liquid chromatography-mass spectrometer and the like.

Meanwhile, examples of problems with the above-mentioned top-down method include not being suitable for trace analyses due to requiring a large amount of protein used on the order of several tens of micrograms, the heavy burden associated with acquiring samples from the body, the possibility of the target protein being overlooked during image analyses in the case the spot of the target protein is in close proximity to the spot of another protein or the case of the luminescence of the target protein being relatively low, and fluctuations in analytical values between individual analyses or technicians as a result of much of the work currently being performed manually and the optimum conditions of area of the gel that is cut out varying according to the amount of protein, thereby resulting in difficulties in quantitative analysis of trace protein components.

In contrast, the bottom-up method is a technique for acquiring information on an expressed protein by carrying out digestive treatment of the entire proteins extracted from cells into peptide fragments using digestive enzymes such as trypsin for which the cleavage site in the amino acid sequence of a protein is known, followed by simultaneously separating and analyzing the peptide fragments with a liquid chromatography-mass spectrometer and comparing with a peptide database possessed by the mass spectrometry system (Non-Patent references 3 and 4).

In the above-mentioned bottom-up method, although detection sensitivity is very high, making this method suitable for trace analyses of several micrograms or less of protein since peptide fragments obtained by digesting protein axe detected with a mass spectrometer. Meanwhile, since the detected components are peptides and not proteins, although qualitative information such as microscopic changes in structure within the protein is obtained with high precision, this method has the problem of difficulty in quantitative analysis of protein per se. As a result thereof, the top-down method is primarily used in quantitative analyses of proteins expressed in trace amounts contained in living tissue and/or biological fluid.

On the basis thereof, Imai, who is one of the joint inventors of the present invention, previously invented novel techniques for quantifying and identifying expressed trace proteins by carrying out fluorogenic derivatization treatment on proteins and peptides expressed in trace amounts in living tissue and/or biological fluid and then detecting and separating the proteins and peptides with high sensitivity by HPLC including fluorescence detection, followed by analyzing the fractions by mass spectrometry (Patent reference 1).

The above-mentioned invention is a superior technology capable of quantitative and qualitative analyses with high precision as a result of sequentially separating and eluting proteins expressed in tissue by liquid chromatography using a porous particulate-packed column and quantifying with a fluorescence detector followed by analyzing protein components by mass spectrometry. On the other hand, as a result of verifying the size of the separation column examined for use in analyzing samples, research results were reported indicating that the optimum size is a column having an inner diameter of 4.6 to 6 mm (Non-patent reference 5).

However, in the case of, for example, carrying out analyses for about 10 hours at a liquid flow rate of an HPLC pump of 0.5 ml/min, the above-mentioned prior art has limitations on the number of specimens that can be analyzed by repeated analysis or continuous analysis using the same sample since this technology consumes about 300 mL of eluent and uses about 8 μg of sample protein for one measurement, while also incurring high analysis costs as a result of using a large amount of high-purity eluent. Thus, there are several problems in development from the research stage to social implementation.

### PRIOR ART REFERENCES

### [Patent References]

[Patent reference 1] Kazuhiro Imai, Japanese Patent No. 4558297, “Title: METHOD OF DETECTING/SEPARATING/IDENTIFYING EXPRESSED TRACE PROTEIN/PEPTIDE” and Kazuhiro Imai, Japanese Patent No. 4679368, “Title: METHOD OF DETECTING/SEPARATING/IDENTIFYING EXPRESSED TRACE PROTEIN/PEPTIDE”
[Patent reference 2] Kazuhiro Imai, Japanese Patent No. 4679368, “Title: METHOD OF DETECTION, SEPARATION AND IDENTIFICATION FOR EXPRESSED TRACE PROTEIN/PEPTIDE”
[Patent reference 3] Kazuhiro Imai, US 2008/280316 (A1), Method of Detection, Separation and Identification for Expressed Trace Protein/Peptide
[Patent reference 4] Kazuhiro Imai, EP 1705482 (A2), Method of Detection, Separation and Identification for Expressed Trace Protein/Peptide

### [Non-Patent References]

[Non-Patent reference 1] Large-scale proteomic for the early detection and therapy tailoring of cancers, Tetushi Yamada, Cytometry Research 20(1) pp. 1-5, 2010
[Non-Patent reference 2] Analytical Techniques for Proteomics-Introduction to Proteome Analysis, Hisasi Hirano, Bunseki, July 2005, pp. 348-353.
[Non-Patent reference 3] New Trends in Shotgun Proteomics, Yasushi Ishihama, Seikagaku, Vol. 83, No. 5, pp. 413-418, May 2011
[Non-Patent reference 4] Online parallel accumulation-serial fragmentation (PASEF) with a novel trapped ion mobility mass spectrometer, Florian Meier, Andreas-David Brunner, Scarlet Koch, Heiner Koch, Markus Lubeck, Michael Krause, Niels Goedecke, Jens Decker, Thomas Kosinski, Melvin A. Park, Nicolai Bache, Ole Hoerning, JUrgen Cox, Oliver Rather, Matthias Mann, Molecular & Cellular Proteomics (2018), Vol. 17, Issue 12, 2534-2545
[Non-Patent reference 5] Efficient chromatographic separation of intact proteins derivatized with a fluorogenic reagent for proteomics analysis, Tomoko Ichibangase, Itaru Yazawa and Kazuhiro Imai, Biomed. Chromatogr. (2013), 27, 1520-1523)

Reducing the size of the column used to separate proteins and peptides and decreasing the pump flow rate of solvents used for analysis can be considered in order to implement the above-mentioned prior art at low cost.

Meanwhile, as a result of verifying the highly precise separation of proteins using a porous particulate-packed column having an inner diameter of 4.0 mm to 6.0 mm during the course of development of the prior art, it is indicated that the larger the inner diameter of the column, the higher precision separation is achieved.

Consequently, since it was difficult to achieve both highly precise protein separation and a decrease in pump flow rate by reducing the inner diameter of the separation column, it has been difficult to provide an apparatus for repetitively and continuously analyzing proteins and peptides present in living tissue and/or biological fluid.

### SUMMARY OF THE INVENTION

### Subject to be Solved by the Invention

With the foregoing in view, as a result of extensive examination of a capillary column having a narrow inner diameter during the course of proceeding with research and development targeted at realization of the object of the prior art, namely reducing the inner diameter of the separation column while retaining highly precise separation of proteins and peptides present in living tissue and/or biological fluid, the inventors of the present invention found that a capillary column utilizing monolithic silica developed by the inventors of the present invention is capable of highly precise detection and separation of proteins in a liquid chromatography system consisting of the configuration shown in FIGS. 1 and 2, thereby leading to completion of the present invention (FIG. 3).

An object of the present invention is to provide a novel analytical method using fingerprints on the basis of the types and expression levels of expressed trace proteins and/or peptides contained in living tissue and/or biological fluid, which is capable of highly precise detection of expressed trace proteins by highly precise protein separation and reduction of column inner diameter as a result of using a monolithic silica capillary column (having, for example, an inner diameter (I.D.) of 0.1 mm and length of 250 mm or 700 mm) developed by the present inventors and a specific fluorogenic reagent (such as DAABD-Cl).

In addition, an object of the present invention is to provide a novel analytical apparatus using fingerprints on the basis of the types and expression levels of expressed trace proteins and/or peptides contained in living tissue and/or biological fluid, which is capable of highly precise detection of expressed trace proteins and/or peptides contained in living tissue and/or biological fluid, for which highly precise separation and highly sensitive detection were difficult with the prior art, as well as highly sensitive detection of expressed trace proteins and/or peptides by reducing capillary diameter, using the above-mentioned monolithic silica capillary column.

The reason for the attainment of precise separation of proteins with a narrow capillary column in contrast to the results by the use of porous particulate-packed columns of the prior art is that, since the thickness of the adsorption layer of the developed porous silica monolith is 1 μm or less and the size of porous particulate-packed fillers currently available commercially has a minimum thickness of 1.5 μm, highly precise separation of proteins, which was unable to be achieved with porous particulate-packed columns, was realized with this capillary column utilizing monolithic silica.

On the basis of the above-mentioned results, the inventors of the present invention solved the problems of the prior art by using a capillary column containing a filler including an adsorption layer with a thickness of 1 μm or less, and invented the analytical method and apparatus according to the present invention that use fingerprints on the basis of the types and expression levels of expressed trace proteins and/or peptides contained in living tissue and/or biological fluid, which had previously been difficult to realize.

The present invention is composed of the following technical means in order to solve the above-mentioned problems.

The present invention relates to an analytical method using fingerprints on the basis of the types of an expression levels of expressed trace proteins and/or peptides contained in living tissue and/or biological fluid for obtaining a profile on the basis of the types and expression levels of proteins and/or peptides expressed in the living body by detecting and separating proteins and/or peptides using the proteins and/or peptides having a fluorescent labelled marker as a target sample followed by continuously quantitatively measuring separated fluorescent labelled proteins and/or peptides by a liquid transfer system capable of transferring liquid at any gradient by combining at least two pumps and a liquid chromatograph provided with a separation column having an inner diameter of 30 to 300 μm and a length of 25 cm or more and containing an adsorbent, the separation column including an adsorption layer with a thickness of 1 μm or less, the adsorption layer containing a silica base material, and a fluorescence detector.

In addition, the present invention relates to an apparatus that uses the analytical method using fingerprints, wherein the apparatus comprises as constituents thereof a liquid transfer system capable of transferring liquid at any gradient with at least two pumps and a liquid chromatography column provided with a separation column having an inner diameter of 30 to 300 μm and a length of 25 cm or more and containing an adsorbent, the separation column including an adsorption layer with a thickness of 1 μm or less, the adsorption layer containing a silica base material, a fluorescence detector, wherein the apparatus is configured to carry out analysis by an analysis method using fingerprints on the basis of the types and expression levels of expressed trace proteins and/or peptides in living tissue and/or biological fluid by supplying as target sample the expressed trace proteins and/or peptides contained labeled with a fluorogenic reagent to the liquid chromatography column provided with a fluorescence detector.

The liquid flow rate of the pumps used in the above-mentioned analytical method and apparatus using fingerprints according to the present invention is preferably 0.1 to 10 μL/min, more preferably 0.3 to 3 μL/min, and can be suitably set to any intended value within these ranges.

The separation column used in the above-mentioned analytical method and apparatus using fingerprints according to the present invention preferably has an inner diameter within the range of 30 to 300 μm and more preferably 50 to 200 μm, and a size such that the column length is preferably 25 cm or more and more preferably 50 to 100 cm.

In addition, the adsorbent packed inside the separation column used in the above-mentioned analytical method and apparatus using fingerprints according to the present invention includes, for the base material thereof, monolithic silica or monolithic polymer in which the thickness of the adsorption layer is 1 μm or less, and silica particles or polymer particles having a porous surface layer in which the thickness of the absorbent layer is 1 μm or less, and has an adsorbent ligand, preferably having carbon chain with 1 to 18 carbon atoms thereof.

In addition, the liquid transfer system used in the above-mentioned analytical method and apparatus using fingerprints according to the present invention is a liquid transfer system capable of transferring liquid at any intended gradient over a long period of time by combining at Least two pumps, and operates at a liquid flow rate over a range of 0.1 to 10 μL/min and preferably over a range of 0.3 to 3 μL/min.

Moreover, the biological component targeted for analysis by the above-mentioned analytical method and apparatus using fingerprints according to the present invention consists of proteins and/or peptides extracted from a human, animal, plant, insect or microorganism and the like, the total weight of the proteins and/or peptides is 5 μg or less and preferably 0.1 to 1 μg, and a fluorescent marker is bound to a thiol group or amino group of the proteins and/or peptides.

The present invention is characterized by labelling a fluorescent marker to proteins extracted from cells and quantitatively analyzing a protein and/or peptide profile with a fluorescence detector. According to the present invention, since only those compounds having a thiol group can be detected, the present invention is suitable for quantitative analysis of proteins and/or peptides. Since the present invention is able to realize searching and examination of proteins and/or peptides related to cell abnormalities, the present invention is useful for diagnosis, drug development and checking for signs of cell abnormalities and the like.

Examples of the fluorogenic reagent for analyzing proteins and/or peptides used in the present invention include the fluorogenic reagents shown in FIG. 8 (such as DAABD-Cl or SBD-F) and these are suitably heated as necessary. Although a property of this fluorogenic reagent (such as DAABD-Cl or SBD-F) is, for example, fluorogenic labeling proteins and/or peptides preferably for 10 to 300 minutes and more preferably 60 to 180 minutes at 30° C. to 100° C. and preferably 40° C. to 70° C., a preferable example of reaction conditions consists of reacting for 10 minutes at 40° C. at an excitation wavelength of 395 nm and fluorescence (detection) wavelength of 505 nm.

Here, a chromatogram is obtained by applying the fluorogenic fluorescent labeled reaction solution to reversed-phase partition HPLC, ion exchange column HPLC or gel filtration HPLC provided with a fluorescence detector.

If a profile differs (if there is any difference) according to the type of cell on the basis thereof, a difference can be determined to be present by a simple comparison. For example, when pathologies are different, in the present invention, as described in the prior art (Patent reference 1 and 2), separation HPLC/fluorescence detection including expressed proteins and/or peptides is performed, intensities of fluorescence peaks are compared to separate fluorescent labelled derivatives of the target expressed proteins and/or peptides, and the resulting target expressed trace proteins and/or peptides are identified by mass spectrometry, MS/MS analysis, database inquiry or structural analysis of the peak fractions of the above-mentioned proteins and/or peptides.

In the present invention, proteins and/or peptides are fluorogenic-labeled by, for example, heating for preferably 10 to 300 minutes and more preferably for 60 to 180 minutes at preferably 30° C. to 100° C. and more preferably 40° C. to 70° C. as was previously described. Subsequently, nearly the entire amount of the reaction solution or a portion thereof can be applied to reversed-phase partition HPLC, ion exchange column HPLC or gel filtration HPLC provided with a fluorescence detector to separate the peak fractions while monitoring fluorescence. In this case, fluorescence detection is set to a wavelength corresponding to the excitation and fluorescence wavelengths of the labeled fluorescent substance.

Next, in providing an explanation of identification of DAABD-derivatized proteins and/or peptides, those substances derivatized with the above-mentioned fluorogenic reagent consisting of vasopressin, oxytocin, somatostatin, calcitonin and amylin were attempted to be identified by LC-MS. The molecular weights thereof are indicated below.

### m/z 541.8 (M+3H)³⁺ [DAABD-vasopressin]

516.0 (M+3H)³⁺ [DAABD-oxytocin]

726.6 (M+3H)³⁺ [DAABD-somatostatin]

989.9 (M+4H)⁴⁺ [DAABD-calcitonin]

892.8 (M+5H)⁵⁺ [DAABD-amylin]

All of these molecular weights represent the molecular weights when DAABD was added to the two cysteine radicals of each peptide, and according to the results of multivalent ion peak detection, during derivatization by DAABD-Cl, the S—S bonds between the cysteine radicals of these peptides were reduced and the reagent was determined to have reacted with both of the two thiol groups.

In addition, in the case of proteins, proteins were able to be identified as a result of attempting to identify by LC-MS/MS detection and database searches using the MASCOT server.

In the present invention, samples containing all types of proteins and/or peptides acquired from the body are targeted for use as target samples. In the method of the present invention, expressed trace proteins and/or peptides present in a target sample are fluorescent labelled with a fluorogenic reagent. In this case, it is important to quantitatively derivatize the expressed proteins and/or peptides by adding a functional group-specific fluorogenic reagent to an aqueous solution of the proteins and/or peptides or, depending on the case, add a surfactant and/or protein denaturing agent.

Namely, in the present invention, expressed proteins and/or peptides are fluorogenic-labeled by adding a surfactant, or depending on the case a reducing agent, to an aqueous solution of the expressed proteins and/or peptides, adding a functional group-specific fluorogenic reagent thereto, and then heating as necessary. In the present invention, a nonionic surfactant, anionic surfactant, cationic surfactant or amphoteric surfactant is used for the above-mentioned surfactant. In addition, in the present invention, although tris(2-carboxyethyl)phosphine or tributyl phosphine is preferably used for the above-mentioned reducing agent, the reducing agent is not limited thereto, but rather any reducing agent having a similar effect can be used in the same manner.

In the present invention, a preferable example of the above-mentioned functional group-specific fluorogenic reagents is the fluorogenic reagent indicated below (FIG. 8).
(1) DAABC-Cl [4-(dimethylaminoethyl aminosulfonyl)-7-chloro-2,1,3-benzoxadiazole)

Although an example of a fluorogenic reagent used in the present invention is DAABD-Cl (4-(dimethylaminoethyl aminosulfonyl)-7-chloro-2,1,3-benzoxadiazole], the fluorogenic reagent is not limited thereto, but rather a fluorogenic reagent that is specific for thiol groups in the form of SBD-F [ammonium 7-fluoro-2,1,3-benzoxadiazole-4-sulfonate] can be used to derivatize Cys-containing trace proteins.

In the present invention, the following fluorogenic reagents, for example, can be suitably used corresponding to the types, objectives of analysis and the like of the expressed proteins and/or peptides. Furthermore, these fluorogenic reagents can be synthesized in the same manner as the methods specifically described in the examples of examined patent publication (B2) of Japanese Patent No. 4558297 relating to the prior art.
(1) TAABD-Cl (7-chloro-2,1,3-benzoxadiazole-4-sulfonylaminoethyl trimethylammonium chloride)
(2) DAABL-F [4-(dimethylaminoethyl aminosulfonyl)-7-fluoro-2,1,3-benzoxadiazole)
(3) TAABD-F (7-fluoro-2,1,3-benzoxadiazole-4-sulfonylaminoethyl trimethylammonium chloride)
(4) DAABSeD-Cl (4-(dimethylaminoethyl aminosulfonyl)-7-chloro-2,1,3-benzoselenadiazole)
(5) TAABSeD-Cl (7-chloro-2,1,3-benzoselenadiazole-4-sulfonylaminoethyl trimethylammonium chloride)
(6) DAABSeD-F (4-(dimethylaminoethyl aminosulfonyl)-7-fluoro-2,1,3-benzoselenadiazole)
(7) TAABSeD-F (7-fluoro-2,1,3-benzoselenadiazole-4-sulfonylamninoethyl trimethylamnonium chloride)
(8) DAABThD-Cl (4-(dimethylaminoethyl aminosulfonyl)-7-chloro-2,1,3-benzothiadiazolej
(9) TAABThD-Cl (7-chloro-2,1,3-benzothiadiazole-4-sulfonylaminoethyl trimethylammonium chloride)
(10) DAABThD-F [4-(dimethylaminoethyl aminosulfonyl)-7-fluoro-2,1,3-benzothiadiazole]
(11) TAABThD-F (7-fluoro-2,1,3-benzothiadiazole-4-sulfonylaminoethyl trimethylammonium chloride)

In addition, the analytical method according to the present invention that uses fingerprints on the basis of types and expression levels of expressed trace proteins and/or peptides contained in living tissue and/or biological fluid enables identification and structural determination of expressed proteins and/or peptides using a mass spectrometer after having detected the expressed proteins and/or peptides with a fluorescence detector followed by fractionating with a fraction collector and the like and subjecting the proteins and/or peptides to fragmentation treatment using a digestive enzyme such as trypsin in the same manner as the techniques described in Patent references 1-4 of the prior art.

During identification and structural analysis of expressed trace proteins and/or peptides contained in living tissue and/or biological fluid according to the present procedure, all or a portion of the expressed proteins and/or peptides can be identified and the structures thereof can be determined by fractionating all ox a portion of the eluate after having been passed through a separation column.

Moreover, since the transfer volume of solvent required for sample separation is allowed to be extremely low, a technique can be used in the liquid chromatography system and separation column used in the present invention in which column eluate can be introduced directly into a mass spectrometer, and after having fractionated the column eluate corresponding to the type of analytical software of the mass spectrometer used, the fractions are subjected to enzymatic digestion followed by identification and structural analysis of eluted components with a mass spectrometer, or a technique can be used in which the column eluate is introduced directly into a mass spectrometer to identify and structurally analyze the eluted components.

As a result of employing the above-mentioned specific configuration, the present invention demonstrates the extraordinary actions and effects described in the description as indicated below.

1) According to the analytical method and apparatus using fingerprints on the basis of the types and expression levels of expressed trace proteins and/or peptides contained in living tissue and/or biological fluid according to the present invention, a profile of expressed trace proteins and/or peptides contained in living tissue or biological fluid can be obtained with a nano-liquid chromatograph having a fluorescence detector by introducing a cysteine-selective fluorogenic reagent into the diverse and trace amounts of expressed proteins and/or peptides contained in living tissue and/or biological fluid.

2) In addition, since the range of the pump liquid transfer flow rate used during measurement is 0.1 to 10 μL/min, even after having measured continuously for 24 hours, the amount of solvent used is low at 0.14 to 14 mL, thereby enabling continuous measurement over the course of several days.

3) Consequently, the status of living tissue and/or biological fluid can be analyzed, or in other words, changes and characteristics of living tissue and the like on the basis of internal and external factors can be determined, by continuously measuring multiple specimens of living tissue and/or biological fluid from different origins or measuring samples of biological fluid intermittently extracted from a culture tank and the like, and analyzing differences in the resulting profiles.

4) Moreover, profiles can be depicted while focusing on protein components related to biological activity in particular, and the amount of time required for analyses relating to changes and characteristics of living tissue and the like on the basis of internal and external factors can be shortened by operating apparatuses in parallel for multiple protein components.

### BRIEF DESCRIPTION OF THE DRAWINGS

FIG. 1 indicates a schematic diagram (apparatus overview) of an apparatus configuration according to an embodiment of the present invention;

FIG. 2 indicates a schematic diagram (parallel apparatus overview) of apparatuses arranged in parallel according to an embodiment of the present invention;

FIG. 3 indicates a highly precise separation diagram on the basis of the results of batch separation of human proteins with a monolithic silica capillary column used in an embodiment of the present invention;

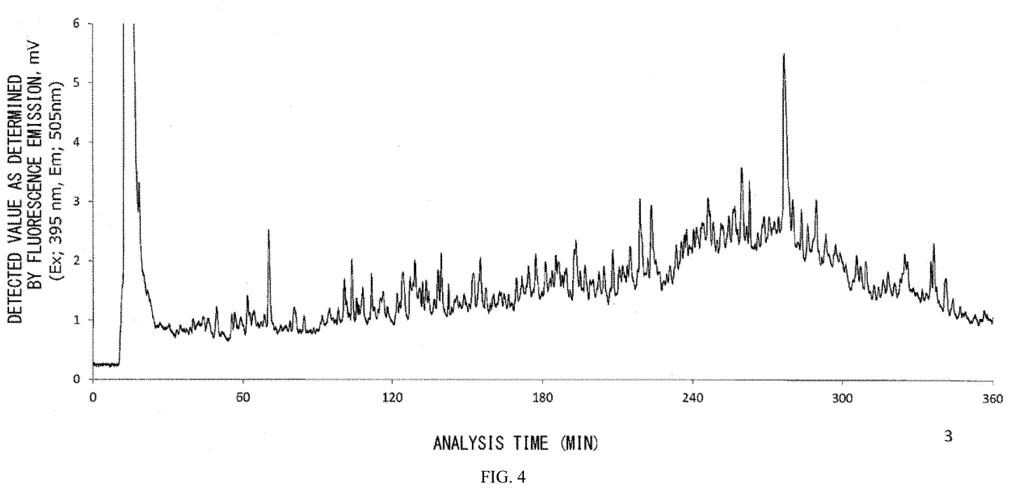

FIG. 4 indicates a graph representing the results of a separation profile of fluorogenic-labeled proteins derived from yeast obtained in Example 1 (analysis example);

FIGS. 5A and 5B indicate graphs representing the results of separation profiles of fluorogenic-labeled proteins derived from yeast obtained in Example 2 (effect of length);

FIGS. 6A to 6C indicate graphs representing the results of separation profiles of fluorogenically-labeled proteins derived from yeast obtained in Example 3 (optimization of gradient time);

FIGS. 7A to 7C indicate graphs representing the results of separation profiles of fluorogenically-labeled proteins derived from yeast obtained in Example 4 (parallel system operation); and

FIG. 8 indicates fluorogenic reagents for protein analysis, DAABD-Cl and SBD-F.

### DESCRIPTION OF THE PREFERRED EMBODIMENTS

Although the following provides an explanation of embodiments of the present invention referring to figures (apparatus configuration examples, test examples or examples and the like), the present invention is not limited in any way by the following configuration examples, test examples or examples and the like.

### Apparatus Configuration Example 1

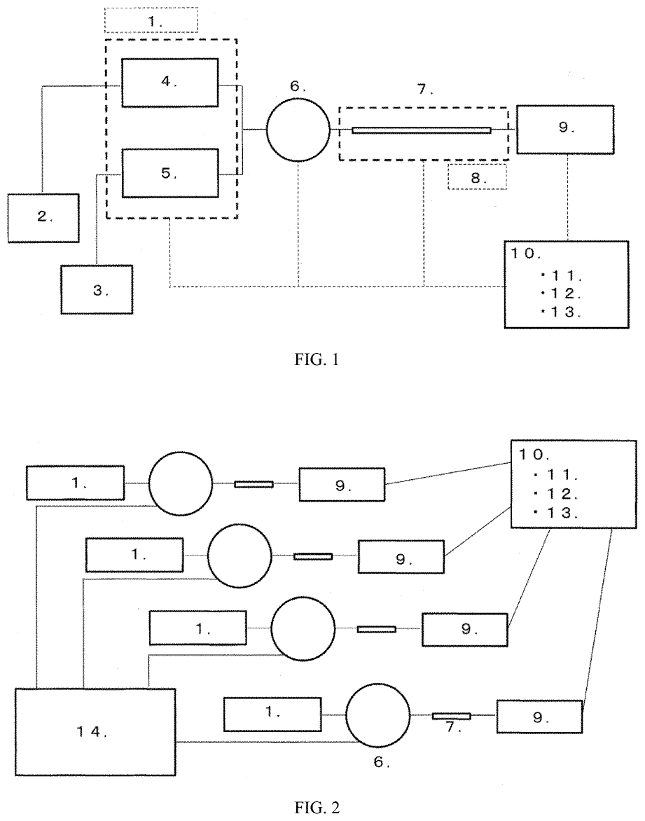

An example of the configuration of an apparatus using the analytical method according to the present invention that uses fingerprints on the basis of the types and expression levels of proteins and/or peptides is explained in detail on the basis of FIG. 1.

An analytical apparatus used in the present analytical method was constructed by coupling a liquid transfer unit capable of transferring liquid at any intended gradient over a long period of time by combining at least two liquid transfer pumps A and B of a mobile phase A and a mobile phase B, a separation column having an inner diameter of 30 to 300 μm and length of 25 cm or more and containing an adsorbent for which the thickness of the adsorption layer containing a silica base material is 1 μm or less, and a nano-liquid chromatography column provided with a fluorescence detector.

The above-mentioned separation column provided with a fluorescence detector was coupled to the above-mentioned liquid transfer unit via a sample injection switching valve and a thermostatic chamber capable of controlling temperature was provided in the separation column. The above-mentioned liquid transfer unit, sample injection switching valve, separation column and fluorescence detector were coupled to a personal computer for system control to construct a basic configuration for acquiring data, carrying out analyses and the like.

Each of the constituents containing a high-performance liquid chromatograph provided with a fluorescence detector (FD-LC) is as indicated below.

LC system: UltiMate 3000 Nano (Thermo Fisher Scientific), detector: FP-2020 Plus with 30 μm I.D. capillary cell (JASCO, Ex: 395 nm, Em: 505 nm), columns (monolithic silica capillary column: 0.1 mm I.D.×250 mm, length: 700 mm; Ultron C4 and Phenyl Column (Shinwa Chemical))

### Apparatus Configuration Example 2

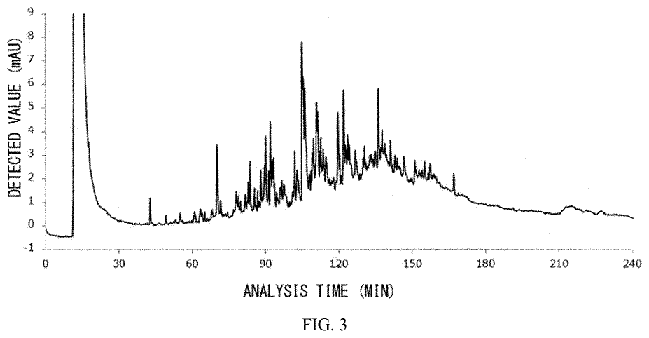

An example of the configuration of parallel apparatuses using the analytical method according to the present invention that uses fingerprints on the basis of the types and expression levels of proteins and/or peptides is explained in detail on the basis of FIG. 2.

An analytical apparatus used in the present analytical method was constructed by coupling four liquid transfer units capable of transferring liquid at any intended gradient over a long period of time by combining at least two liquid transfer pumps, separation columns having an inner diameter of 30 to 300 m and length of 25 cm or more and containing an adsorbent for which the thickness of the adsorption layer containing a silica base material is 1 μm or more, and nano-liquid chromatography columns provided with fluorescence detectors via sample injection switching valves.

The mobile phase of a sample was allowed to be injected automatically by coupling an automatic sample injection apparatus (auto sampler) to the above-mentioned liquid transfer units via the above-mentioned sample injection switching valves. Apparatuses for acquiring data and carrying out analyses and the like were provided in parallel by coupling each of the above-mentioned fluorescence detectors to a personal computer for system control to construct a basic configuration. Furthermore, each of the constituents containing a high-performance liquid chromatograph provided with a fluorescence detector (FD-LC) was the same as in Apparatus Configuration Example 1.

### Test Example 1

A fluorogenic reagent (fluorogenic derivatization reagent) was synthesized in the present test example.

### Synthesis of DAABD-Cl

4-chlorosulfonyl-7-chloro-2,1,3-benzoadiazole (CBD-Cl, 126.53 mg) was dissolved in CH₃CN followed by dropping in N,N-dimethylethylenediamine and adding triethylamine. After stirring for about 10 minutes at room temperature, the reaction liquid was dried under reduced pressure followed by purifying with a silica gel column (CH₂Cl₂) to obtain 4-(dimethylaminoethyl aminosulfonyl)-7-chloro-2,1,3-benzoxadiazole (DAABD-Cl, 20.2 mg, 87.4%).

Confirmation data of the resulting compound is indicated below.

¹H-NMR (CD₃OD): 7.94 (1H, d, J=7.5), 7.65 (1H, d, J=7.5), 3.06 (2H, t, J=6.7), 2.30 (2H, t, J=6.7), 2.02 (6H, s); ESI-MS: m/z 305 (M+H)⁺

### Test Example 2

Sensitivities during MS of fluorogenic reagents (fluorogenic derivatization reagents) were compared in the present test example.

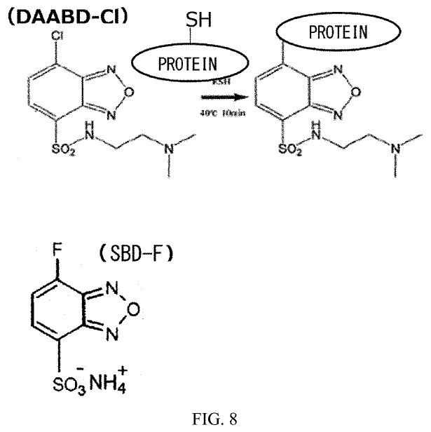

The sample prepared in the above-mentioned synthesis example detected by LC-MS and not labeled with a fluorogenic derivatization reagent along with that derivatized with SBD-F (FIG. 8) were compared on the basis of relative intensity.

Relative intensities on the basis of a value of 1 for the respective heights of cysteine, homocysteine, and GHS not labeled with fluorogenic derivatization reagent were as shown in Table 1 below. Furthermore, although a reaction time of 120 minutes was required when carrying out derivatization at 40° C. in the case of SBD-F, in the case of DAABD-Cl, the reaction was completed in 10 to 20 minutes. Thus, a reaction time of 20 minutes was preferable in the case of DAABD-Cl. On the basis of the above, DAABD-Cl was determined to demonstrate high sensitivity in MS. In addition, since the mobile phase is acidic, DAABD-derivatized derivatives were thought to be positively charged and water soluble.

TABLE 1

| | SBD-F | DAABD-Cl |
|---|---|---|
| cysteine | 23 | 3.0 × 10³ |
| homocysteine | 4.0 | 2.3 × 10² |
| GHS | 1.6 | 2.1 × 10² |

Detection Limit of DAABD-Derivatized Peptides/Proteins

50 μL each of a 10 μM mixture of the ten types of peptide/protein standards listed in the following Table 2, 17.5 mM DAABD-Cl, 10 mM EDTA and 50 mM CHAPS were mixed followed by reacting for 30 minutes at pH 9.0 and 40° C. Furthermore, each reagent was dissolved in 0.10 M borate buffer (pH 9.0) containing 6.0 M guanidine hydrochloride. The DAABD-derivatized peptides/proteins that formed were measured using HPLC and detection limits of fluorescence detection were compared with SBD-F.

TABLE 2

| Peptides and | Molecular | Number of | Detection limit (fmol) | |
|---|---|---|---|---|
| proteins | weight (Da) | cystenyl residues | DAABD-Cl | SBD-F |
| vasopressin | 1084 | 2 | 7.0 | 5.0 |
| oxytocin | 1007 | 2 | 4.5 | 1.3 |
| somatostatin | 1638 | 2 | 20 | 1.8 |
| calcitonin | 3418 | 2 | 5.0 | 6.0 |
| amylin (rat) | 3920 | 2 | 4.5 | 1.2 |
| insulin | 5808 | 6 | 2.2 | 0.7 |
| α-acid | 21547 | 4 | 8.5 | 1.3 |
| glycoprotein | | | | |
| α-lactalbumin | 16228 | 8 | 3.5 | 0.5 |
| albumin (BSA) | 66385 | 35 | 0.5 | 0.2 |
| leptin | 16014 | 2 | 30 | 3.0 |

Next, a detailed explanation of the present invention is provided on the basis of examples thereof.

### Example 1

A system was constructed using the following apparatus configuration and analysis conditions in order to prepare a profile using proteins extracted from yeast.

System Configuration
Liquid transfer pump, auto sampler, column oven: UltiMate 3000 Nano (Thermo Fisher Scientific)
Fluorescence detector: FP-2020 (JASCO)
Detection cell: Capillary cell (inner diameter: 30 μm, cell length: 1 cm)
Column: C4 modified monolithic silica capillary column (inner diameter: 100 μm, length: 70 cm)

Chromatographic Separation Conditions
Mobile phase A: 0.1% TFA-ultrapure water
Mobile phase B: 0.1% TFA-acetonitrile
Liquid transfer flow rate: 0.5 μL/min
Gradient conditions: 20% to 35% B (360 min)
Column temperature: 50° C.
Fluorescence wavelengths: Excitation wavelength (Ex) 395 nm, detection wavelength (Em) 505 nm, gain 1000
Sample volume: 0.5 μL
Sample weight: 0.5 μg

The analysis sample was prepared under the following conditions according to the patent literature.

### Preparation of Analysis Sample

Protein extracted from Saccharomyces cerevisiae (trade name: MS Compatible Yeast Protein Extract, Intact, Promega) was used for the sample. The yeast is composed of 12 million bases and roughly 6,000 genes are involved in protein expression.

After adding 6 M guanidine solution (600 μL), 100 mM EDTA-6 M guanidine solution (200 μL), 10 mM TCEP-6 M guanidine solution (50 μL) and 140 mM DAABD-C-acetonitrile solution (50 μL) to 1 mg of the sample and allowing to react for 10 minutes at 40° C., 10% TFA (30 μL) was added while cooling with ice, resulting in the obtaining of 1 mg/mL solution of fluorogenic-labeled protein.

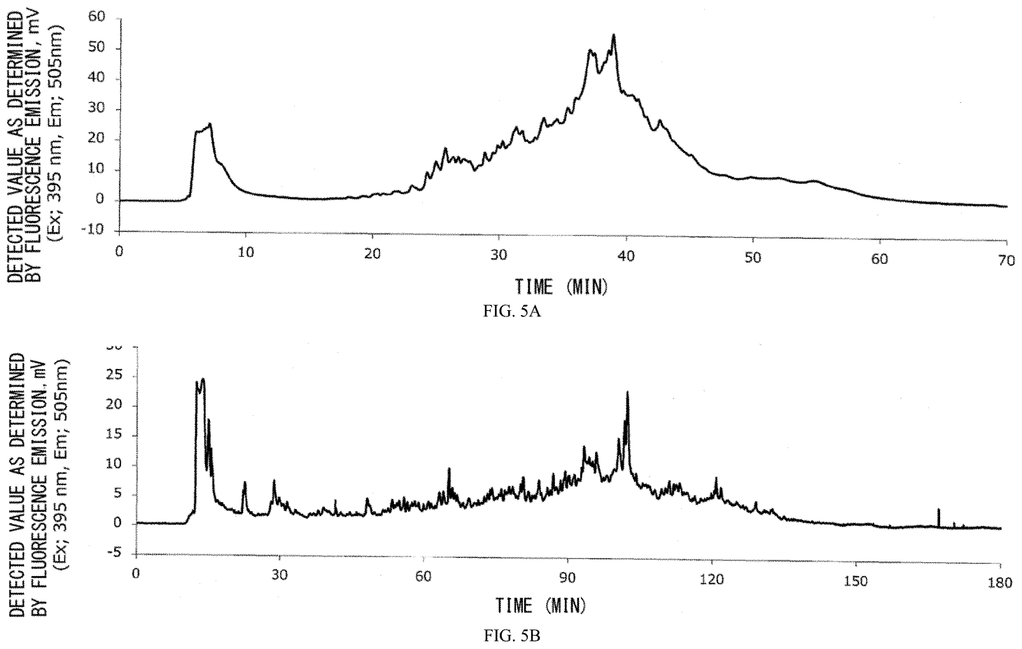

The results of analyzing over the course of six hours are shown in FIG. 4. The vertical axis of the graph indicates detected values (mV) as determined by fluorescence emission. As a result, in addition to obtaining about 350 peaks according to the type of protein, a profile was obtained that is derived from proteins expressed by the yeast of the sample (genome size: 12.1 million bases, number of genes: 6,275). The amount of solvent used in this measurement was about 0.2 mL.

### Example 2

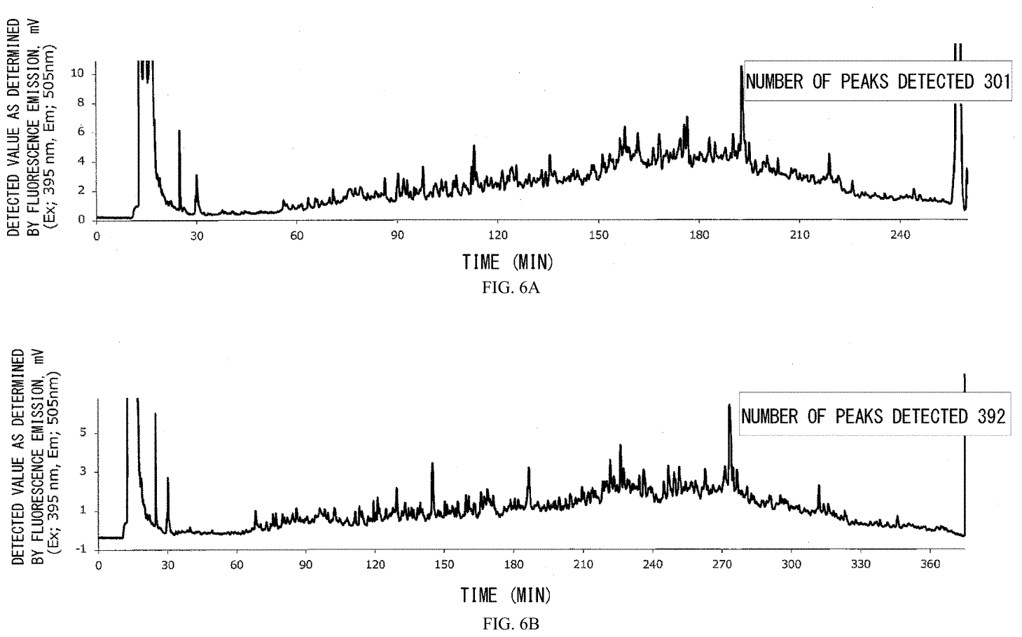

Columns having lengths of 25 cm and 70 cm were used and analyses were carried out for analysis times suitable for each length in order to confirm the effect of separation column length on profile. The analysis conditions consisted of changing only the gradient conditions, using a gradient of 10% to 55% B (60 min) for the column having a length of 25 cm and a gradient of 20% to 55% B (240 min) for the column having a length of 70 cm. The results are shown in FIGS. 5A and 5B. The vertical axis of the graph indicates detected values (mV) as determined by fluorescence emission.

FIG. 5A is the profile obtained for the 25 cm column while FIG. 5B is the profile obtained for the 70 cm column. The profile obtained with the 25 cm column of FIG. 5A is not considered to exhibit favorable separation in comparison with the profile obtained with the 70 cm column of FIG. 5B. On the other hand, both profiles can be seen to exhibit similar shapes.

In the analytical method using fingerprints on the basis of the types and expression levels of expressed trace proteins and/or peptides contained in living tissue and/or biological fluid, extremely high-precision separation is not necessarily required. In addition, since species having a small number of genes have a small number of protein types, a column length suitable for that species can be selected. The results of Example 2 indicate that the present invention enables the selection of column length that is suitable for the particular species.

### Example 3

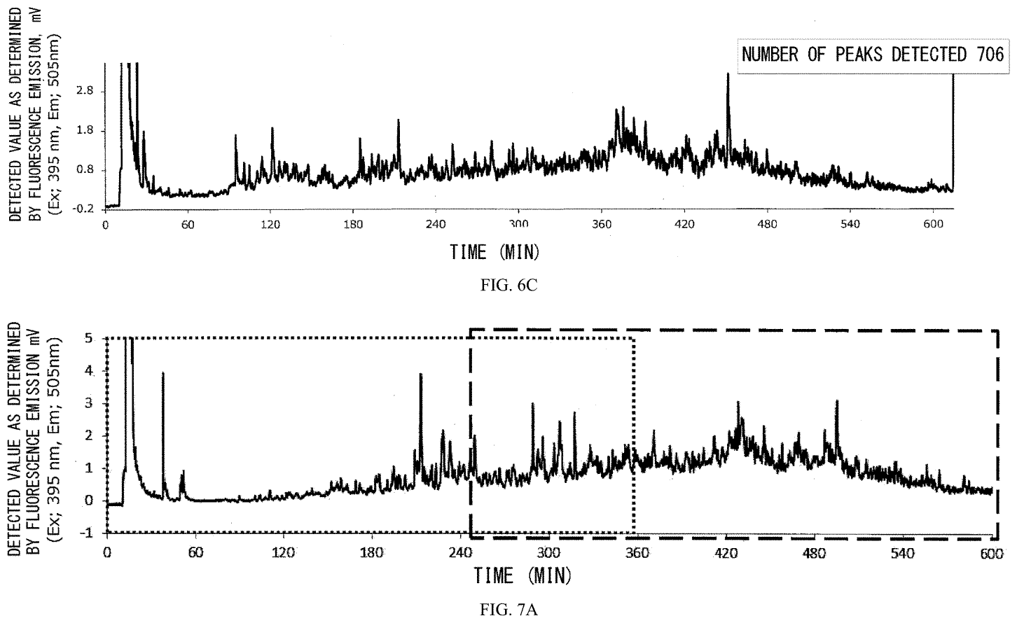

The relationship between separation time and the number of protein and/or peptide peaks detected was verified in an attempt to obtain more precise protein and/or peptide information. The results are shown in FIGS. 6A to 6C. The vertical axis of the graph indicates detected values (mV) as determined by fluorescence emission. The analysis conditions consisted of using respective gradient of 25% to 45% for 240 minutes, 360 minutes and 600 minutes. As a result, the numbers of peaks detected were 301, 392 and 706, respectively.

Since a greater number of protein peaks were detected as the gradient time increased, longer and gentler gradient conditions were indicated to be suitable for obtaining a more precise protein profile. Thus, the present invention was demonstrated to be useful with respect to being able to be stably operated for a longer period of time in the case of, for example, searching for disease markers targeted at humans having a larger number of proteins.

### Example 4

It was presumed from Example 3 that gradient analysis for several tens of hours is required for a more precise fingerprint analysis of species predicted to have a large number of genes and express a large number of proteins and/or peptides such as in the case of humans.

On the other hand, since disease-specific profiles and disease-related proteins and/or peptides are expected to be discovered by comparing the profiles of multiple specimens, the above-mentioned analytical method according to the present invention is required to analyze multiple specimens in a single day. The operation of multiple apparatuses in parallel was verified in order to realize highly precise analyses in a short period of time by the above-mentioned analytical method and apparatus using fingerprints according to the present invention.

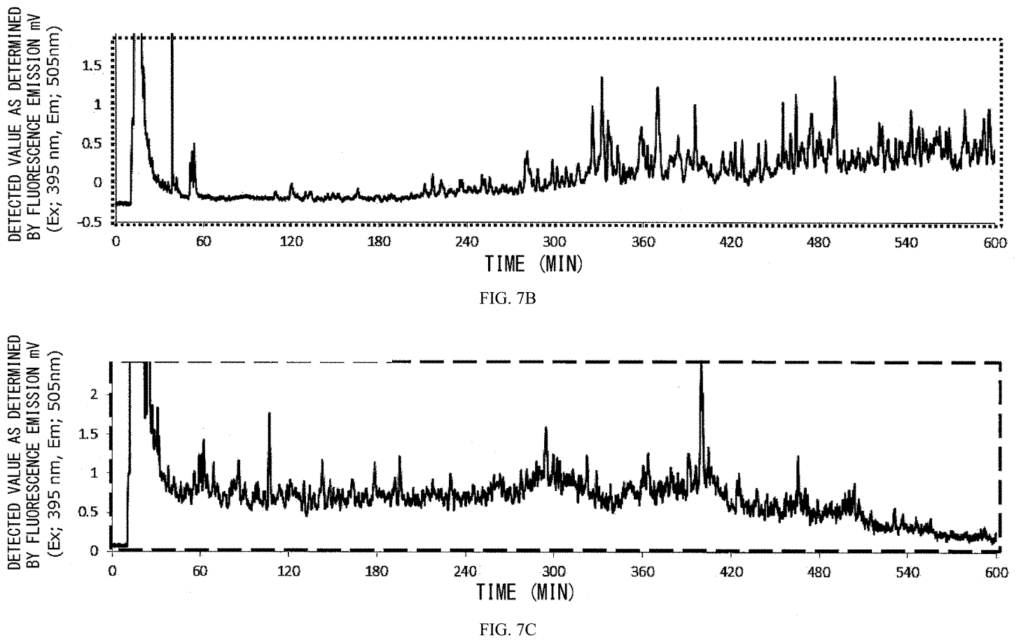

FIG. 7A is a profile obtained by applying gradient conditions consisting of 20% to 45% B for an analysis time of 600 minutes. In contrast, a profile obtained by applying gradient conditions consisting of 20% to 35% B for the same analysis time is shown in FIG. 7B, while a profile obtained by applying gradient conditions consisting of 30% to 45% B for the same analysis time is shown in FIG. 7C.

Gradient conditions consisting of 20% to 35% B of FIG. 7B resulted in more precise separation corresponding to the 0 to 360 minutes of FIG. 7A while retaining the same profile. In addition, gradient conditions consisting of 30% to 45% B of FIG. 7C achieved more precise separation corresponding to the 240 to 600 minutes of FIG. 7A while retaining the same profile.

In this manner, since proteins and/or peptides are eluted and detected corresponding to the concentration of the eluent in accordance with the principle of liquid chromatography, profiles corresponding to the concentration of the eluent were determined to be obtained even through gradient conditions differed. In addition, more precise separation was achieved since the gradient conditions were gentler.

Thus, as FIG. 2, this system was verified to make it possible to obtain highly precise protein and/or peptide profiles obtained by analyses equivalent to 100 hours in an analysis time of just 10 hours, or obtain analysis results obtained in analyses equivalent to 10 hours in just 1 hour, by operating multiple apparatuses in parallel, such as by using 10 apparatuses, and shifting their respective gradient conditions.

Since the present invention uses a micro column that precisely separates proteins and/or peptides and greatly reduces the consumption of solvent required for analyses, it is realistic to operate this type of system in parallel, and is expected to be widely used in, for example, the diagnosis of diseases in which proteins and/or peptides are involved or prediction of the efficacy of personalized treatment.

As was previously described in detail, the present invention relates to an analytical method and apparatus using fingerprints on the basis of the types and expression levels of expressed trace proteins and/or peptides contained in living tissue and/or biological fluid, and together with providing a novel analytical method using fingerprints on the basis of the types and expression levels of expressed trace proteins and/or peptides expressed in living tissue and/or biological fluid, the present invention provides an analytical method and an analytical apparatus using fingerprints on the basis of the types and expression levels of expressed trace proteins and/or peptides of living tissue and/or biological fluid obtained through continuous analyses of multiple specimens by using nano-liquid chromatography technology. The present invention is useful not only in the fields of medicine and pharmacology, but also in numerous industries ranging from agriculture to food science, such as through improvement of culturing technology using microorganisms or improving microbial gene modification technology towards increased efficiency thereof, by using the above-mentioned analytical method and apparatus using fingerprints.

1. An analytical method using fingerprints on the basis of the types of an expression levels of expressed trace proteins and/or peptides contained in living tissue and/or biological fluid for obtaining a profile on the basis of the types and expression levels of proteins and/or peptides expressed in the living body by detecting and separating proteins and/or peptides using the proteins and/or peptides having a fluorescent marker bound to a target sample followed by continuously quantitatively measuring separated fluorescent labelled proteins and/or peptides by a liquid transfer system capable of transferring liquid at any gradient by combining at least two pumps and a liquid chromatography column provided with a separation column having an inner diameter of 30 to 300 μm and a length of 25 cm or more and containing an adsorbent, the separation column including an adsorption layer with a thickness of 1 μm or less, the adsorption layer containing a silica base material, and a fluorescence detector.

2. The analytical method using fingerprints on the basis of the types and expression levels of expressed trace proteins and/or peptides contained in living tissue and/or biological fluid according to claim 1, wherein the liquid transfer flow rate of the pumps is 0.1 to 10 μL/min.

3. The analytical method using fingerprints on the basis of the types and expression levels of expressed trace proteins and/or peptides contained in living tissue and/or biological fluid according to claim 1, wherein the separation column is a monolithic silica capillary column containing a monolithic silica gel or a monolithic polymer gel.

4. The analytical method using fingerprints on the basis of the types and expression levels of expressed trace proteins and/or peptides contained in living tissue and/or biological fluid according to claim 1, wherein the separation column contains a silica particle or a synthetic polymer particle having a porous surface layer.

5. The analytical method using fingerprints on the basis of the types and expression levels of expressed trace proteins and/or peptides contained in living tissue and/or biological fluid according to claim 1, wherein the number of carbon atoms of an adsorbent ligand for a fluorescent labelled protein bound to the surface of the separation column is 1 to 18.

6. The analytical method using fingerprints on the basis of the types and expression levels of expressed trace proteins and/or peptides contained in living tissue and/or biological fluid according to claim 1, wherein the proteins are proteins extracted from a human, animal, plant, insect, or microorganism, and the total weight of the proteins is 5 μg or less.

7. The analytical method using fingerprints on the basis of the types and expression levels of expressed trace proteins and/or peptides contained in living tissue and/or biological fluid according to claim 1, wherein the fluorescent marker is bound to a thiol group or amino group present in the proteins and/or peptides.

8. The analytical method using fingerprints on the basis of the types and expression levels of expressed trace proteins and/or peptides contained in living tissue and/or biological fluid according to claim 1, wherein multiple protein profiles targeted for analysis are selected, and multiple liquid transfer units including at least two liquid transfer pumps are operated in parallel.

9. An apparatus that uses the analytical method using fingerprints, wherein the apparatus comprises as constituents thereof a liquid transfer system capable of transferring liquid at any gradient with at least two pumps and a liquid chromatography column provided with a separation column having an inner diameter of 30 to 300 μm and a length of 25 cm or more and containing an adsorbent, the separation column including an adsorption layer with a thickness of 1 μm or less, the adsorption layer containing a silica base material, a fluorescence detector, wherein the apparatus is configured to carry out analysis using fingerprints on the basis of the types and expression levels of expressed trace proteins and/or peptides in living tissue and/or biological fluid by supplying as test sample the expressed trace proteins and/or peptides contained labeled with a fluorogenic reagent to the liquid chromatography column provided with a fluorescence detector.

10. The apparatus according to claim 9, which is configured to be operated in parallel by installing in parallel multiple liquid transfer units containing at least two liquid transfer pumps.

11. The apparatus according to claim 9, wherein the separation column is a monolithic silica capillary column containing a monolithic silica gel or a monolithic polymer gel.

12. The apparatus according to any one of claim 9, wherein the adsorbent contains a silica particle or a synthetic polymer particle having a porous surface layer.

13. The apparatus according to claim 9, wherein the adsorbent is subjected to surface modification treatment of the silica base material with an adsorbent ligand or phenyl-form silylating agent having 1 to 18 carbon atoms.
